(19) United States
(12) Patent Application Publication
(10) Pub. No.: US 2012/0167899 A1
(43) Pub. Date: Jul. 5, 2012

(54) Absorbent digital Pad

(76) Inventor: Clarence Dorsey, Houston, TX (US)

(21) Appl. No.: 13/229,568

(22) Filed: Sep. 9, 2011

Related U.S. Application Data
(63) Continuation-in-part of application No. 11/250,389, filed on Oct. 3, 2005, now abandoned.

(51) Int. Cl. A61F 13/06

(52) U.S. Cl. 128/894

(57) ABSTRACT

The present invention relates to a pad to be inserted between the web spaces of human toes. The pad comprise an non-absorbent outer section encompassing an absorbent inner section. The pad can be manufactured within various shapes and sizes.

### NON-PROVISIONAL PATENT

This patent claims the benefit of parent 111250,389. This is a continuation in part of application 111250,389.

### BACKGROUND

This invention relates to an article for placement between toes of a human being to prevent irritation. Athlete's feet is a common ailment that is difficult to cure. Athlete's feet is a fungal infection of the skin that causes scaling flaking and itching of the areas of the foot. The most common of these infections occur in the web spaces in between the toes. Athlete's foot can be treated by a number of pharmaceutical products. The present invention provides an easy method to keep the web spaces in between the toes and allow the infection to heel more quickly.

### SUMMARY

The object of this invention it to provide a pad that is capable of separating the toes of the feet and preventing irritation of the same. Another object of the present invention is to separate the toes and prevent irritation therein. The present invention provides an inter-digital pad of various sizes, shapes and depth to accommodate the web spaces between the toes. The inter-digital pad comprises an outer layer of an impermeable material encompassing an absorbent layer of material. Numerous other objects and advantages will be evident as the following specification is read in conjunction with the accompanying drawings below.

### BRIEF DESCRIPTION OF DRAWINGS

FIG. 1 is a front, side and top view of a thinner elongated strip of absorbent material.

FIG. 2 is a front, side and top view of two shorter and thinner individual absorbent segments being placed directly on top of each other.

FIG. 3 is a front, side and top view of the two shorter and thinner individual absorbent segments, which have been positioned, placed and/or layered directly on top of each other.

FIG. 4 is a front, side and top view of a thicker elongated strip of absorbent material.

FIG. 5 is a front, side and top view of a thicker and longer elongated strip of absorbent material.

FIG. 6 is a front, side and top view of the two shorter and thinner individual absorbent segments positioned, placed and/or layered directly on top of each other and has also been positioned, placed and/or layered directly on top of one longer and thicker elongated strip of absorbent material.

FIG. 7 is a front, side and top view of a thinner elongated strip of absorbent material.

FIG. 7-A is a front, side and top view of a thinner and longer elongated strip of absorbent material

FIG. 8 is a front, side and top view of the two shorter and thinner individual absorbent segments being positioned. placed and/or layered directly on top of each other and also being positioned, placed and/or layered directly on top of one longer and thicker elongated strip of absorbent material and furthered being positioned, placed and/or layered directly on top of one longer and thinner elongated strip of absorbent material.

FIG. 9 is a front, side and top view of the said two shorter and thinner individual absorbent segments which has been positioned, placed and/or layered directly on top of each other and which has also been positioned, placed and/or layered directly on top of one longer and thicker elongated strip of absorbent material and which has also been positioned, placed and/or layered directly on top of one longer and thinner elongated strip of absorbent material whereby the said two longer thicker and thinner elongated strips of absorbent materials.

FIG. 10 is a front, side and top view of the said two shorter and thinner individual absorbent segments which has been positioned, placed and/or layered directly on top of each other and which has also been positioned, placed and/or layered directly on top of one longer and thicker elongated strip of absorbent material and which has also been positioned, placed and/or layered directly on top of one longer and thinner elongated strip of absorbent material whereby the said two longer thicker and thinner elongated strips of absorbent materials is about to be folded directly over and onto the said two shorter thinner individual absorbent segments.

FIG. 11 is a front, side and top view of the said two shorter and thinner individual absorbent segments which has been positioned, placed and/or layered directly on top of each other and which has been also positioned. placed and/or layered directly on top of one longer and thicker elongated strip of absorbent material and which has been also positioned. placed and/or layered directly on top of one longer and thinner elongated strip of absorbent material whereby the said two longer thicker and thinner elongated strips of absorbent materials which has been folded directly over and onto the said two shorter thinner individual absorbent segments and onto the one half ends of two said longer thicker and thinner elongated strips of absorbent material with matching ends to form a backward E-shaped visual representation.

FIG. 12 is a front, side and top view of said the two shorter and thinner individual absorbent segments which has been positioned, placed and/or layered directly on top of each other and which has been also positioned, placed and/or layered directly on top of one longer and thicker elongated strip of absorbent material and which has been also positioned, placed and/or layered directly on top of one longer and thinner elongated strip of absorbent materi whereby the said two longer thicker and thinner elongated strips of absorbent materials which have been also folded directly over and onto the said two shorter thinner individual absorbent segments, whereby the said backward E-shaped in FIG. 11 has been transformed into a predetermined oval shaped impression of a specified length, width.

FIG. 13 is a front, side and top view of said the two shorter and thinner individual absorbent segments which has been positioned, placed and/or layered directly on top of each other and which has been also positioned, placed and/or layered directly on top of one longer and thicker elongated strip of absorbent material and which has been also positioned, placed and/or layered directly on top of one longer and thinner elongated strip of absorbent material whereby the said two longer thicker and thinner elongated strips of absorbent materials which have been also folded directly over and onto the said two shorter thinner individual absorbent segments, in FIG. 12 has been transformed into a “tuft of cotton and/or other material” after being sufficiently compressed and/or sealed or bonded or otherwise affixed around the edges, ends and/or center into an oval shape of any predetermined specified length, width and thickness, including up to approximately one half of an inch (V2 of an inch). This “tuft of cotton and/or other material” is not otherwise compressed in the remaindering areas that are considered by this inventor to be the necessary and/or essential components of such “tuft of cotton and/or other material”.

FIG. 14 is a front, side and top view of an oval shaped digital absorbent pad of any predetermined, self-sustaining, desired shape, length, width and thickness after trimming away and removal of the excess materials.

Alternately, FIG. 15 is a front, side and top view of a thicker elongated absorbent material. FIG. 16 is a front, side and top view of two shorter and thicker individual elongated absorbent segments being placed directly on top of each other.

FIG. 17 is a front, side and top view of the two shorter and thicker elongated strips individual absorbent segments, which has been positioned, placed and/or layered directly on top of each other with matching ends and twisted in the middle of said individual elongated absorbent segments one time.

FIG. 18 is a front, side and top view of the two shorter and thicker elongated strips of individual absorbent segments which has been positioned, placed and/or layered directly on top of each other with matching ends and twisted in the middle of said individual elongated strips of individual absorbent segments one time which is about to be folded directly over and onto the remaindering one half ends of the said two shorter thicker individual elongated strips of individual absorbent segments. If adhesives, including glue is used to seal or bond or otherwise affixed around the edges, ends and/or center of this digit absorbent pad, such process is performed at this stage.

FIG. 19 is a front, side and top view of the four shorter and thicker elongated strips of individual absorbent segments with matching ends which has been folded directly over and onto each other whereby the remaindering one half (2) ends of the said four shorter thicker individual absorbent segments and being sufficiently compressed into a twisted, layered and folded digital absorbent pad of any predetermined specified length, width and thickness ready for use.

FIG. 20 illustrates a top view of an oval shape strip.

FIG. 85 is a side view of a Digital Absorbent pad Flash Drive like shaped with six (6) holes of any predetermined, self-sustaining, desired shape, length, width and thickness after trimming away and removal of the excess materials.

FIG. 86 is a side view of a Digital Absorbent pad K like shaped with three (3) holes of any predetermined, self-sustaining, desired shape, length, width and thickness after trimming away and removal of the excess materials.

FIG. 87 is a side view of a Digital Absorbent pad Spacer like shaped with two (2) holes of any predetermined. self-sustaining, desired shape, length, width and thickness after trimming away and removal of the excess materials.

FIG. 88 is a side view of a Digital Absorbent pad Comb like shaped of any predetermined, self-sustaining, desired shape, length, width and thickness after trimming away and removal of the excess materials.

FIG. 89 is a side view of a Digital Absorbent pad Nut shaped of any predetermined, self-sustaining, desired shape, length, width and thickness after trimming away and removal of the excess materials.

FIG. 90 is a side view of a Digital Absorbent pad Crescent like shaped of any predetermined, self-sustaining, desired shape, length, width and thickness after trimming away and removal of the excess materials.

FIG. 91 is a side view of a Digital Absorbent pad Half Nut shaped of any predetermined, self-sustaining, desired shape, length, width and thickness after rimming away and removal of the excess materials.

FIG. 92 is a side view of a Digital Absorbent pad Strip shaped with one (1) slit of any predetermined, self-sustaining, desired shape, length, width and thickness after trimming away and removal of the excess materials.

FIG. 93 is a side view of a Digital Absorbent pad Strip shaped with one (1) slit and one (1) ridge on both sides of any predetermined, self-sustaining, desired shape, length, width and thickness after trimming away and removal of the excess materials.

FIG. 94 is a side view of a Digital Absorbent pad Strip shaped of any predetermined, self-sustaining, desired shape, length, width and thickness after trimming away and removal of the excess materials.

FIG. 95 is a side view of a Digital Absorbent pad Strip shaped digital absorbent pad with one (1) slit of any predetermined, self-sustaining, desired shape, length, width and thickness after trimming away and removal of the excess materials.

FIG. 96 is a side view of a Digital Absorbent pad Right Angle like shaped digital absorbent pad with three (3) holes of any predetermined, self-sustaining, desired shape, length, width and thickness after trimming away and removal of the excess materials.

FIG. 101 is a side view of a Digital Absorbent pad Funnel like shaped of any predetermined, self-sustaining, desired shape, length, width and thickness after trimming away and removal of the excess materials.

FIG. 102 is a side view of a Digital Absorbent pad Fin like shaped of any predetermined, self-sustaining, desired shape, length, width and thickness after trimming away and removal of the excess materials.

FIG. 103 is a side view of a Digital Absorbent pad Fin like shaped of any predetermined, self-sustaining, desired shape, length, width and thickness after trimming away and removal of the excess materials.

FIG. 104 is a side view of a Digital Absorbent pad Fin like shaped of any predetermined, self-sustaining, desired shape, length, width and thickness after trimming away and removal of the excess materials.

FIG. 105 is a side view of a Digital Absorbent pad Overfold like shaped of any predetermined, self-sustaining, desired shape, length, width and thickness after trimming away and removal of the excess materials.

FIG. 106 is a side view of a Digital Absorbent pad Fold like shaped of any predetermined, self-sustaining, desired shape, length, width and thickness after trimming away and removal of the excess materials.

FIG. 107 is a side view of a Digital Absorbent pad Fold like shaped three (3) holes of any predetermined, self-sustaining, desired shape, length, width and thickness after trimming away and removal of tile excess materials.

FIG. 108 is a side view of a Digital Absorbent pad Fold like shaped of any predetermined, self-sustaining, desired shape, length, width and thickness after trimming away and removal of the excess materials.

FIG. 109 is a side view of a Digital Absorbent pad Fold like shaped with two (2) slits of any predetermined, self-sustaining, desired shape, length, width and thickness after trimming away and removal of the excess materials.

FIG. 110 is a side view of a Digital Absorbent pad Fold like shaped with holes of any predetermined, self-sustaining, desired shape, length, width and thickness after trimming away and removal of the excess materials.

FIG. 111 is a side view of a Digital Absorbent pad Fold like shaped with four (4) holes of any predetermined, self-sustaining, desired shape, length, width and thickness after trimming away and removal of the excess materials.

FIG. 112 is a side view of a Digital Absorbent pad Fold like shaped with three (3) holes of any predetermined, self-sustaining, desired shape, length, width and thickness after trimming away and removal of the excess materials.

FIG. 113 is a side view of a Digital Absorbent pad Roll like shaped of any predetermined, self-sustaining, desired shape, length, width and thickness after trimming away and removal of the excess materials.

FIG. 114 is a side view of a Digital Absorbent pad D like shaped digital absorbent pad of any predetermined, self-sustaining, desired shape, length, width and thickness after trimming away and removal of the excess materials.

FIG. 115 is a side view of a Digital Absorbent pad Elongated like shaped digital absorbent pad with four (4) holes of any predetermined, self-sustaining, desired shape, length, width and thickness after trimming away and removal of the excess materials.

FIG. 116 is a side view of a Digital Absorbent pad Diamond like shaped digital absorbent pad with one (1) slit of any predetermined, self-sustaining, desired shape, length, width and thickness after trimming away and removal of the excess materials.

FIG. 117 is a side view of a Digital Absorbent pad Diamond like shaped with five (5) holes of any predetermined, self-sustaining, desired shape, length, width and thickness after trimming away and removal of the excess materials.

FIG. 118 is a side view of a Digital Absorbent pad Kite like shaped of any predetermined, self-sustaining, desired shape, length, width and thickness after trimming away and removal of the excess materials.

FIG. 119 is a side view of a Digital Absorbent pad Pocket like shaped with three (3) slits of any predetermined, self-sustaining, desired shape, length, width and thickness after trimming away and removal of the excess materials.

FIG. 120 is a side view of a Digital Absorbent pad Pocket like shaped with two (2) slits of any predetermined, self-sustaining, desired shape, length, width and thickness after trimming away and removal of the excess materials.

FIG. 121 is a side view of a Digital Absorbent pad Oblate like shaped with ridges on both sides of any predetermined, self-sustaining, desired shape, length, width and thickness after trimming away and removal of the excess materials.

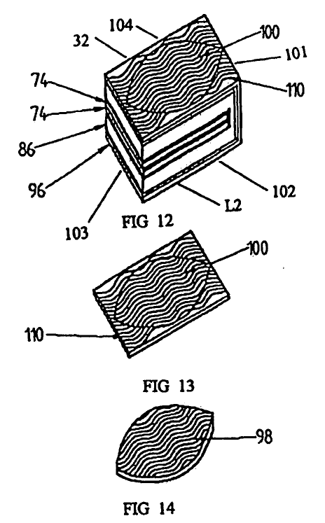

FIG. 12 is a side view of a Digital Absorbent pad Round like shaped with three (3) slits of any predetermined, self-sustaining, desired shape, length, width and thickness after trimming away and removal of the excess materials.

FIG. 123 is a side view of a Digital Absorbent pad round like shaped with three (3) holes of any predetermined, self-sustaining, desired shape, length, width and thickness after trimming away and removal of the excess materials.

FIG. 124 is a side view of a Digital Absorbent pad Egg like shaped of any predetermined, self-sustaining, desired shape, length, width and thickness after trimming away and removal of the excess materials.

FIG. 125 is a side view of a Digital Absorbent pad Skirt shaped of any predetermined, self-sustaining, desired shape, length, width and thickness after trimming away and removal of the excess materials.

FIG. 126 is a side view of a Digital Absorbent pad Antique Casket like shaped of any predetermined, self-sustaining, desired shape, length. width and thickness after trimming away and removal of the excess materials.

FIG. 127 is a side view of a Digital Absorbent pad Hexagon like shaped of any predetermined, self-sustaining, desired shape, length, width and thickness after trimming away and removal of the excess materials.

FIG. 128 is a side view of a Digital Absorbent pad Square like shaped with slits of any predetermined, self-sustaining, desired shape, length, width and thickness after trimming away and removal of the excess materials.

FIG. 129 is a side view of a Digital Absorbent pad Lobe like shaped of any predetermined, self-sustaining, desired shape, length, width and thickness after trimming away and removal of the excess materials.

FIG. 130 is a side view of a Digital Absorbent pad Trough like shaped of any predetermined, self-sustaining, desired shape, length, width and thickness after trimming away and removal of the excess materials.

FIG. 131 is a side view of a Digital Absorbent pad Turbine like shaped with two (2) slits of any predetermined, self-sustaining, desired shape, length, width and thickness after trimming away and removal of the excess materials.

FIG. 132 is a side view of a Digital Absorbent pad Pointed Blade shaped of any predetermined, self-sustaining, desired shape, length, width and thickness after trimming away and removal of the excess materials.

FIG. 133 is a side view of a Digital Absorbent pad Hollow Tooth like shaped of any predetermined, self-sustaining, desired shape, length, width and thickness after trimming away and removal of the excess materials.

FIG. 134 is a side view of a Digital Absorbent pad Spiral Helical shaped of any predetermined, self-sustaining, desired shape, length, width and thickness after trimming away and removal of the excess materials.

FIG. 135 is a side view of a Digital Absorbent pad Scroll like shaped of any predetermined, self-sustaining, desired shape, length, width and thickness after trimming away and removal of the excess materials.

FIG. 136 is a side view of a Digital Absorbent pad Fossa/Pit like shaped digital absorbent pad of any predetermined, self-sustaining, desired shape, length, width and thickness after trimming away and removal of the excess materials.

FIG. 137 is a side view of a Digital Absorbent pad Flower like shaped of any predetermined, self-sustaining, desired shape, length, width and thickness after trimming away and removal of the excess materials.

FIG. 138 is a side view of a Digital Absorbent pad Basketball like shaped of any predetermined, self-sustaining, desired shape, length, width and thickness after trimming away and removal of the excess materials.

FIG. 139 is a side view and top view of a Digital Absorbent pad Strip like shaped with three (3) ridges on both sides of any predetermined, self-sustaining, desired shape, length, width and thickness after trimming away and removal of the excess materials.

FIG. 140 is a side view and top view of a Digital Absorbent pad Strip like shaped with a ridge on both sides of any predetermined, self-sustaining, desired shape, length, width and thickness after trimming away and removal of the excess materials.

FIG. 141 is a side view of a Digital Absorbent pad Loops like shaped digital absorbent pad of any predetermined, self-sustaining, desired shape, length, width and thickness after trimming away and removal of the excess materials.

FIG. 142 is a side view of a Digital Absorbent pad Coil like shaped of any predetermined, self-sustaining, desired shape, length, width and thickness after trimming away and removal of the excess materials.

FIG. 143 is a side view of a Digital Absorbent pad Tears like shaped of any predetermined, self-sustaining, desired shape, length, width and thickness after trimming away and removal of the excess materials.

FIG. 144 is a side view of a Digital Absorbent pad Strip like shaped with right indentation of any predetermined, self-sustaining, desired shape, length, width and thickness after trimming away and removal of the excess materials.

FIG. 145 is a side view of a Digital Absorbent pad Fold like shaped of any predetermined, self-sustaining, desired shape, length, width and thickness after trimming away and removal of the excess materials.

FIG. 146 is a side view of a Digital Absorbent pad Overfold like shaped digital absorbent pad of any predetermined. self-sustaining, desired shape, length, width and thickness after trimming away and removal of the excess materials.

FIG. 147 is a side view of a Digital Absorbent pad Strip like shaped with bottom indentation and ridges on both sides of any predetermined, self-sustaining, desired shape, length, width and thickness after trimming away and removal of the excess materials.

FIG. 148 is a side view of a Digital Absorbent pad Pocket like shaped with woven materials of any predetermined, self-sustaining, desired shape, length, width and thickness after trimming away and removal of the excess materials.

FIG. 149 is a side view of a Digital Absorbent pad Slab Tear like shaped (material tom up and put back together) of any predetermined, self-sustaining, desired shape, length, width and thickness after trimming away and removal of the excess materials.

FIG. 150 is a side view of a Digital Absorbent pad Lines/Curve like shaped of any predetermined, self-sustaining, desired shape, length, width and thickness after trimming away and removal of the excess materials.

FIG. 151 is a side view of a Digital Absorbent pad Funnel like shaped with one (1) slit and one (1) ridge on both sides of any predetermined, self-sustaining, desired shape, length, width and thickness after trimming away and removal of the excess materials.

FIG. 152 is a side view of a Digital Absorbent pad Sheet like shaped with a curved top and ridges on both sides of any predetermined, self-sustaining, desired shape, length, width and thickness after trimming away and removal of the excess materials.

FIG. 153 is a side view of a Digital Absorbent pad Wrapped Trous like shaped of any predetermined, self-sustaining, desired shape, length, width and thickness after trimming away and removal of the excess materials.

FIG. 154 is a side view of a Digital Absorbent pad Wrapped Torus like shaped with slits and ridges on both sides of any predetermined, self-sustaining, desired shape, length, width and thickness after trimming away and removal of the excess materials.

FIG. 155 is a side view of a Digital Absorbent pad Tangled like shaped of any predetermined, self-sustaining, desired shape, length, width and thickness after trimming away and removal of the excess materials.

FIG. 156 is a side view of a Digital Absorbent pad Roll like shaped of any predetermined, self-sustaining, desired shape, length, width and thickness after trinuning away and removal of the excess materials.

FIG. 157 is a side view of a Digital Absorbent pad Leaf like shaped of any predetermined, self-sustaining, desired shape, length, width and thickness after trimming away and removal of the excess materials.

FIG. 158 is a side view of a Digital Absorbent pad Ellipse like Tear shaped of any predetermined, self-sustaining, desired shape, length, width and thickness after trimming away and removal of the excess materials.

FIG. 159 is a side view of a Digital Absorbent pad Tear shaped of any predetermined, self-sustaining, desired shape, length, width and thickness after trimming away and removal of the excess materials.

FIG. 160 is a side view of a Digital Absorbent pad Tear Slab like shaped of any predetermined, self-sustaining, desired shape, length, width and thickness after trimming away and removal of the excess materials.

FIG. 165 is a side view of a Digital Absorbent pad Pointed Blade like shaped with one (1) slit of any predetermined, self-sustaining, desired shape, length, width and thickness after trimming away and removal of the excess materials.

FIG. 166 is a side view of a Digital Absorbent pad Bullet like shaped of any predetermined, self-sustaining, desired shape, length, width and thickness after trimming away and removal of the excess materials.

FIG. 167 is a side view of a Digital Absorbent pad Turbine like shaped with ridges on both sides and slits of any predetermined, self-sustaining, desired shape, length, width and thickness after trimming away and removal of the excess materials.

FIG. 168 is a side view of a Digital Absorbent pad Coil like shaped with one (1) straight line of any predetermined, self-sustaining, desired shape, length, width and thickness after trimming away and removal of the excess materials.

FIG. 169 is a side view of a Digital Absorbent pad Wrap like shaped of any predetermined, self-sustaining, desired shape, length, width and thickness after trimming away and removal of the excess materials.

FIG. 170 is a side view of a Digital Absorbent pad Wrap like shaped digital with one (1) strip with one slit and ridges on both sides and slits of any predetermined, self-sustaining, desired shape, length, width and thickness after trimming away and removal of the excess materials.

FIG. 171 is a side view of a Digital Absorbent pad Backward D like shaped with encased materials of any predetermined, self-sustaining, desired shape, length, width and thickness after trimming away and removal of the excess materials.

FIG. 172 is a side view of a Digital Absorbent pad Square like shaped with slits and ridges on both sides and slits of any predetermined, self-sustaining, desired shape, length, width and thickness after trimming away and removal of the excess materials.

FIG. 173 is a side view of a Digital Absorbent pad Overfold like shaped of any predetermined, self-sustaining, desired shape, length, width and thickness after trimming away and removal of the excess materials.

FIG. 174 is a side view of a Digital Absorbent pad U like shaped of any predetermined, self-sustaining, desired shape, length, width and thickness after trimming away and removal of the excess materials.

FIG. 175 is a side view of a Digital Absorbent pad an Angle like shaped with slits and ridges on both sides of any predetermined, self-sustaining, desired shape, length, width and thickness after trimming away and removal of the excess materials.

FIG. 176 is a side view of a Digital Absorbent pad an horizontal slab like shaped with slits and ridges on both sides of any predetermined, self-sustaining, desired shape, length, width and thickness after trimming away and removal of the excess materials.

FIG. 177 is a side view of a Digital Absorbent pad invaginate like shaped with slit of any predetermined, self-sustaining, desired shape, length, width and thickness after trimming away and removal of the excess materials.

FIG. 178 is a side view of a Digital Absorbent pad Horizontal Strip like shaped with slit like curves of any predetermined, self-sustaining, desired shape, length, width and thickness after trimming away and removal of the excess materials.

FIG. 179 is a side view of a Digital Absorbent pad Horizontal Strip like shaped with one slit of any predetermined, self-sustaining, desired shape, length, width and thickness after trimming away and removal of the excess materials.

FIG. 180 is a side view of a Digital Absorbent pad Coil like shaped of any predetermined, self-sustaining, desired shape, length, width and thickness after trimming away and removal of the excess materials.

FIG. 181 is a side view of a Digital Absorbent pad Round like shaped of any predetermined, self-sustaining, desired shape, length, width and thickness after trimming away and removal of the excess materials.

FIG. 182 is a side view of a Digital Absorbent pad Cone like shaped of any predetermined, self-sustaining, desired shape, length, width and thickness after trimming away and removal of the excess materials.

FIG. 183 is a side view of a Digital Absorbent pad Cube like shaped of any predetermined, self-sustaining, desired shape, length, width and thickness after trimming away and removal of the excess materials.

FIG. 184 is a side view of a Digital Absorbent pad Crescent like shaped of any predetermined, self-sustaining, desired shape, length, width and thickness after trimming away and removal of the excess materials.

FIG. 185 is a side view of a Digital Absorbent pad 0 like shaped of any predetermined, self-sustaining, desired shape, length, width and thickness after trimming away and removal of the excess materials.

FIG. 186 is a side view of a Digital Absorbent pad E like shaped with slits of any predetermined, self-sustaining, desired shape, length, width and thickness after trimming away and removal of the excess materials.

FIG. 187 is a side view of a Digital Absorbent pad T like shaped of any predetermined, self-sustaining, desired shape, length, width and thickness after trimming away and removal of the excess materials.

FIG. 188 is a side view of a Digital Absorbent pad L like shaped of any predetermined, self-sustaining, desired shape, length, width and thickness after trimming away and removal of the excess materials.

FIG. 189 is a side view of a Digital Absorbent pad P like shaped any predetermined, self-sustaining, desired shape, length, width and thickness after trimming away and removal of the excess materials.

FIG. 190 is a side view of a Digital Absorbent pad Q like shaped of any predetermined, self-sustaining, desired shape, length, width and thickness after trimming away and removal of the excess materials.

FIG. 191 is a side view of a Digital Absorbent pad D like shaped with an angle like shaped digital absorbent pad of any predetermined, self-sustaining, desired shape, length, width and thickness after trimming away and removal of the excess materials.

FIG. 192 is a side view of a Digital Absorbent pad E like shaped with slits and ridges on both sides of any predetermined, self-sustaining, desired shape, length, width and thickness after trimming away and removal of the excess materials.

FIG. 193 is a side view of a Digital Absorbent pad Pepper like shaped with slits and ridges on both sides of any predetermined, self-sustaining, desired shape, length, width and thickness after trimming away and removal of the excess materials.

FIG. 194 is a side view of a Digital Absorbent pad Half Pepper like shaped of any predetermined, self-sustaining, desired shape, length, width and thickness after trimming away and removal of the excess materials.

FIG. 195 is a side view of a Digital Absorbent pad Horn like shaped with one (1) slit of any predetermined, self-sustaining, desired shape, length, width and thickness after trimming away and removal of the excess materials.

FIG. 196 is a side view of a Digital Absorbent pad R like shaped with one (1) slit of any predetermined, self-sustaining, desired shape, length, width and thickness after trimming away and removal of the excess materials.

FIG. 197 is a side view of a Digital Absorbent pad Octagon like shaped of any predetermined, self-sustaining, desired shape, length, width and thickness after trimming away and removal of the excess materials.

FIG. 198 is a side view of a Digital Absorbent pad Seal Head & Neck like shaped of any predetermined, self-sustaining, desired shape, length, width and thickness after trimming away and removal of the excess materials.

FIG. 199 is a side view of a Digital Absorbent pad Heptagon like shaped of any predetermined, self-sustaining, desired shape, length, width and thickness after trimming away and removal of the excess materials.

FIG. 200 is a side view of a Digital Absorbent pad Cylinder like shaped of any predetermined, self-sustaining, desired shape, length, width and thickness after trimming away and removal of the excess materials.

FIG. 201 is a side view of a Digital Absorbent pad Heart Like shaped of any predetermined, self-sustaining, desired shape, length, width and thickness after trimming away and removal of the excess materials.

FIG. 202 is a side view of a Digital Absorbent pad D like shaped with ridges on both sides of any predetermined, self-sustaining, desired shape, length, width and thickness after trimming away and removal of the excess materials.

FIG. 203 is a side view of a Digital Absorbent pad Partial Upside Down Heart like shaped with indentation of any predetermined, self-sustaining, desired shape. length, width and thickness after trimming away and removal of the excess materials.

FIG. 204 is a side view of a Digital Absorbent pad Knife like shaped of any predetermined, self-sustaining, desired shape, length, width and thickness after trimming away and removal of the excess materials.

FIG. 205 is a side view of a Digital Absorbent pad Pick like shaped of any predetermined, self-sustaining, desired shape, length, width and thickness after trimming away and removal of the excess materials.

FIG. 206 is a side view of a Digital Absorbent pad Pick like shaped of any predetermined, self-sustaining, desired shape, length, width and thickness after trimming away and removal of the excess materials.

FIG. 207 is a side view of a Digital Absorbent pad Horn like shaped of any predetermined, self-sustaining, desired shape, length, width and thickness after trimming away and removal of the excess materials.

FIG. 208 is a side view of a Digital Absorbent pad Horn shaped of any predetermined, self-sustaining, desired shape, length, width and thickness after trimming away and removal of the excess materials.

FIG. 209 is a side view of a Digital Absorbent pad Pick like shaped with slit and ridge on both sides of any predetermined, self-sustaining, desired shape, length, width and thickness after trimming away and removal of the excess materials.

FIG. 210 is a side view of a Digital Absorbent pad Horn like shaped of any predetermined, self-sustaining, desired shape, length, width and thickness after trimming away and removal of the excess materials.

FIG. 211 is a side view of a Digital Absorbent pad Seal Head & Neck like shaped of any predetermined, self-sustaining, desired shape, length, width and thickness after trimming away and removal of the excess materials.

FIG. 212 is a side view of a Digital Absorbent pad Horn like shaped with one (1) slit and small ridge on both sides of any predetermined, self-sustaining, desired shape, length, width and thickness after trimming away and removal of the excess materials.

FIG. 213 is a side view of a Digital Absorbent pad C-Shaped or Heel Tap like shaped designed, constructed and assembled on a device and inserted in between the toes of any predetermined, self-sustaining, desired shape, length, width and thickness after trimming away and removal of the excess materials.

FIG. 214 is a side view of a Digital Absorbent pad of an oval like shaped designed, constructed and assembled on a device and inserted in between the toes of any predetermined, self-sustaining, desired shape, length, width and thickness after trimming away and removal of the excess materials.

FIG. 215 is a side view of a Digital Absorbent pad of a Overfold Long Strip like shaped inserted of any inserted in a weaving type manner of any predetermined, self-sustaining, desired shape, length, width and thickness after trimming away and removal of the excess materials.

FIG. 216 is a side view of a Digital Absorbent pad of a Long Strip like shaped inserted in a weaving type manner of any predetermined, self-sustaining, desired shape, length, width and thickness after trimming away and removal of the excess materials.

### DETAILED DESCRIPTION

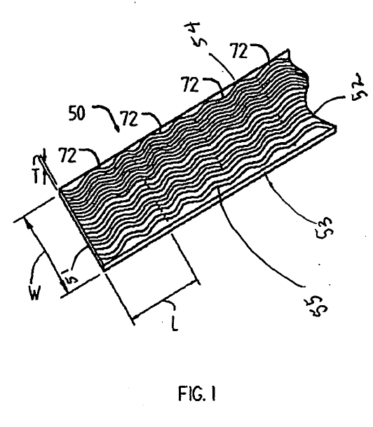

Referring to FIG. 1 there is shown one embodiment of the present invention, inter-digital pad (50). In the illustrated embodiment in FIG. 1, inter-digital pad (50) has a polygonal shape with an elongated length constructed to fit the length of the longer toes of a human foot. Inter-digital pad (50) is defined by a top side (55), front edge (51), a back edge (52) and two opposing side edges (54, 55). In the embodiment illustrated in FIG. 1, the front edge (51) is linear. back edge (52) is curved, and topside (55) has a plurality of ridges (72) incorporated thereon. The pad can be manufactured and segmented into any length required to be inter-disposed, between the web spaces of the toes.

Each inter-digital pad (50) has a length equivalent to the length between each toe, a width to encompass the opposing surface areas between each toe, and a depth to accommodate the space between each toe. When inserted, the back edge (52) can have a curved edge in one embodiment to seat upon the space between a toe. When inter-digital pad is inter-deposed between the toes, opposing side edges (54, 55) is pressed upon opposing surface areas.

Referring to FIG. IA, there is shown a cross-section of inter-digital pad 50. As shown, inter-digital pad comprises an inner section (57) composed of an absorbent material encompassed by an outer section (59) made of a non-absorbent material with the outer peripheral edges sealed. When in use, the moisture between the web spaces of the toes are absorbed through the outer section (59) and contained within the inner section (57) allowing the web spaces between the toes to be maintain dry. Additionally, a medicated product can be contained within inner section (57) and/or outer section (59).

The inter-digital pad 50 inner section can be made of cotton, polyester, sponge material, rayon, cellulose material, absorbent polymers, any combination thereof, or any other suitable absorbent material. The inter-digital pad 50 outer section can be made of non-absorbent polymers, gel material, glass material, synthetic material, or any other suitable non-absorbent material. The combination can range from 1 to 99. Additionally, each of the various shapes of the inter-digital pad can be made with a plurality of apertures within the outer section of inter-digital pad or a plurality of apertures extending through both the inner section and outer section of the inter-digital pad (50). The pad can be up to 8 inches in width, 10 inches in length, or 4 inches in thickness. FIG. 1B illustrates the pad seated between the web spaces of the toes. FIG. 1C illustrates a cross section of the pad seated between one toe.

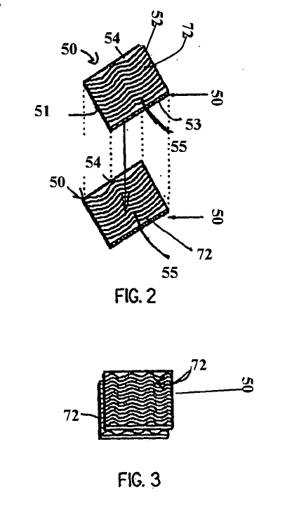

Referring to FIG. 2 there is shown another embodiment of the present invention, inter-digital pad (50). In this illustrated embodiment, back edge (52) is linear and inter-digital pad has a shorter length to accommodate toes of shorter length.

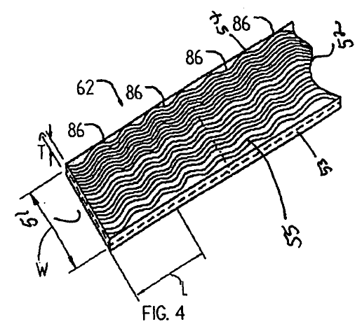

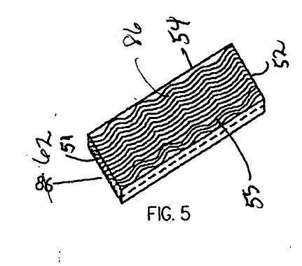

Referring to FIG. 3 there is shown a plurality of inter-digital pads (50) overlaying each other for insertion between the web spaces of toes of larger depth. The embodiment illustrated in FIG. 4 is substantially the same as the illustrated embodiment in FIG. 1 except that this of inter digital pad (62) embodiment has a thicker depth. As illustrated the front edge (51) is linear, back edge (52) is curved, underside is flat, and topside (55) has ridges.

Referring to FIG. 5 there is shown another embodiment of the present invention, inter-digital pad (86). this embodiment is the substantially the same as FIG. 4 except it has all straight edges.

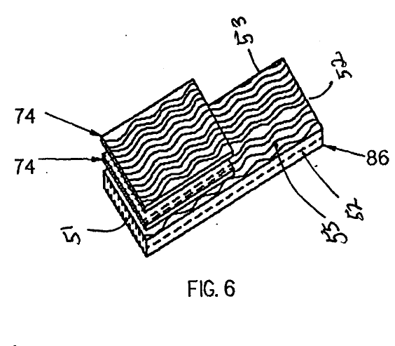

Referring to FIG. 6 there is shown another embodiment of the present invention, inter-digital pad (70). In this embodiment the various embodiments of inter-digital pads illustrated in FIG. 3 are stack atop the embodiment illustrated in FIG. 5. These pads are pads (74) are stack atop a thicker pad (86).

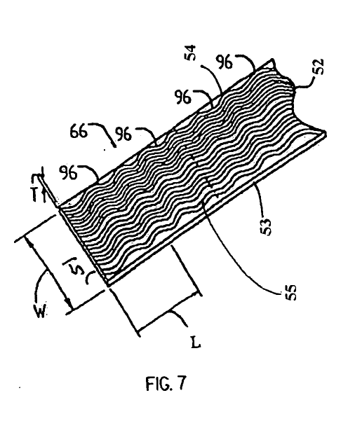

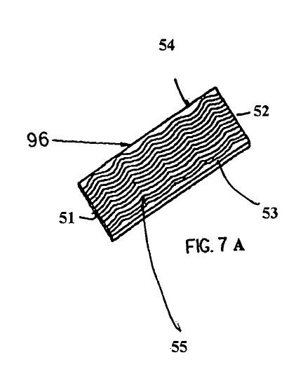

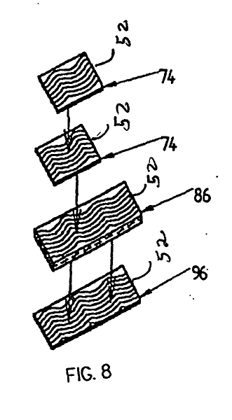

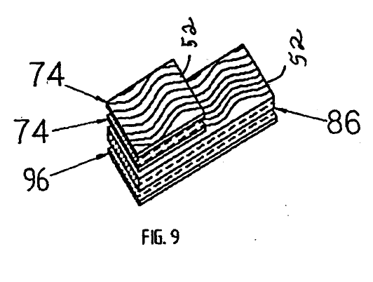

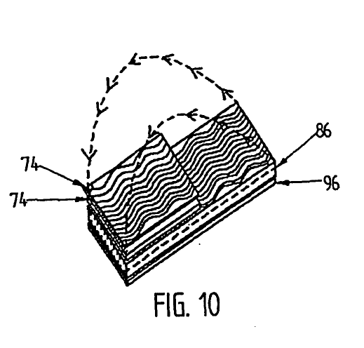

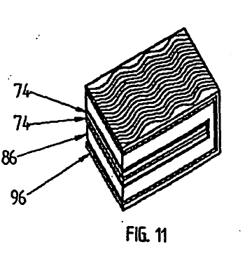

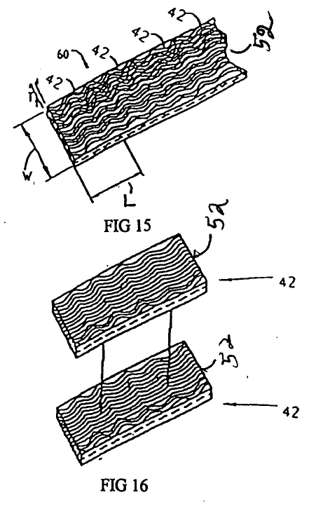

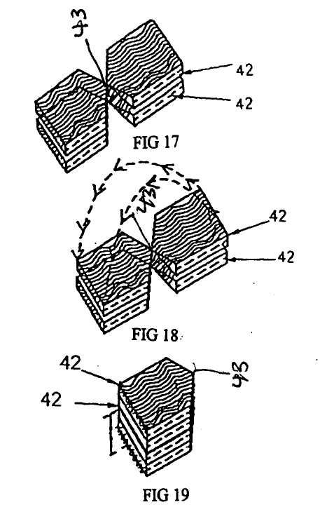

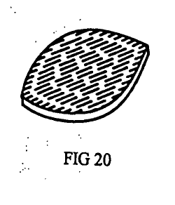

FIG. 7-20 illustrates alternative configurations of the inter-digital pad. FIG. 7 shows an alternative embodiment with deeper and longer curved ridges on top side. FIG. 7A depicts an embodiment of the pad with a straight back side edge with deeper and longer curved ridges on the top side. FIG. 8 depicts an embodiment showing layering of thin shorter and longer pads. FIG. 9 depicts an embodiment showing layering of thicker and shorter pads. FIG. 10 depicts an embodiment illustrating longer and shorter pad of a thicker depth layered together; the pads also have longer ridges on the top surface of both the longer and shorter pads. FIG. 11 depicts an embodiment with longer pads folder over forming a recessed portion for receiving shorter pads there between. FIG. 12 depicts the same embodiment illustrated in FIG. 11, but an oval shape is incorporated into the top side of the longer pad having a polygonal shape. FIG. 13 depicts an embodiment of the pad with an oval incorporated therein. FIG. 14 illustrates an embodiment of the pad in an oval shape. FIG. 15 depicts an embodiment of the pad with twisted ridges incorporated upon the top side of the pad. FIG. 16 depicts an embodiment with illustrates double edge layers. FIG. 17-18 depicts an embodiment composed of opposing triangular shaped pad with connecting apex points. FIG. 18 depicts the range of layering. FIG. 19 depicts the two pads layered upon each other. FIG. 20 illustrates an alternative embodiment of the inter-digital pad in an oval shape.

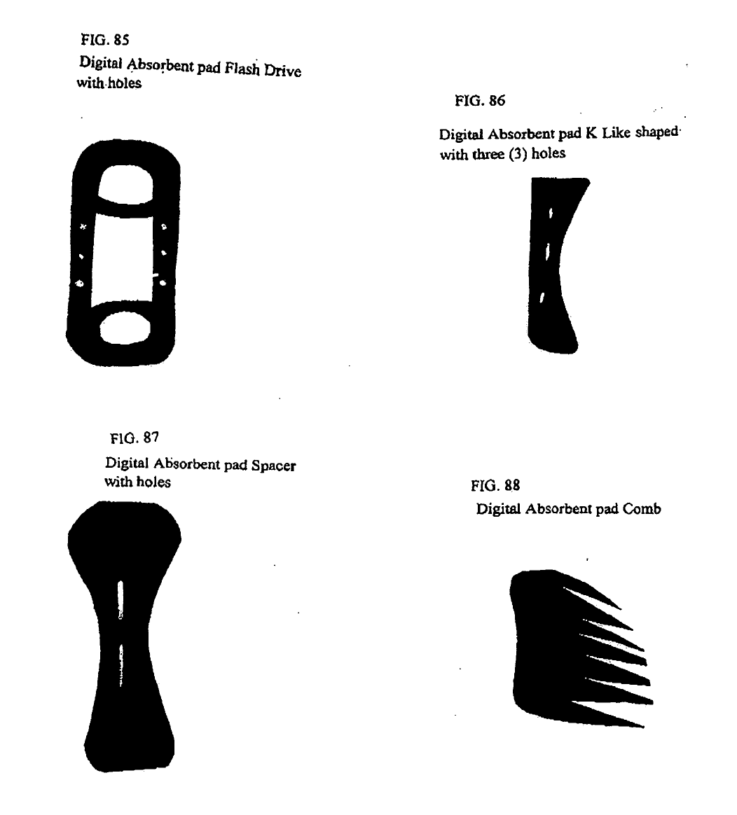

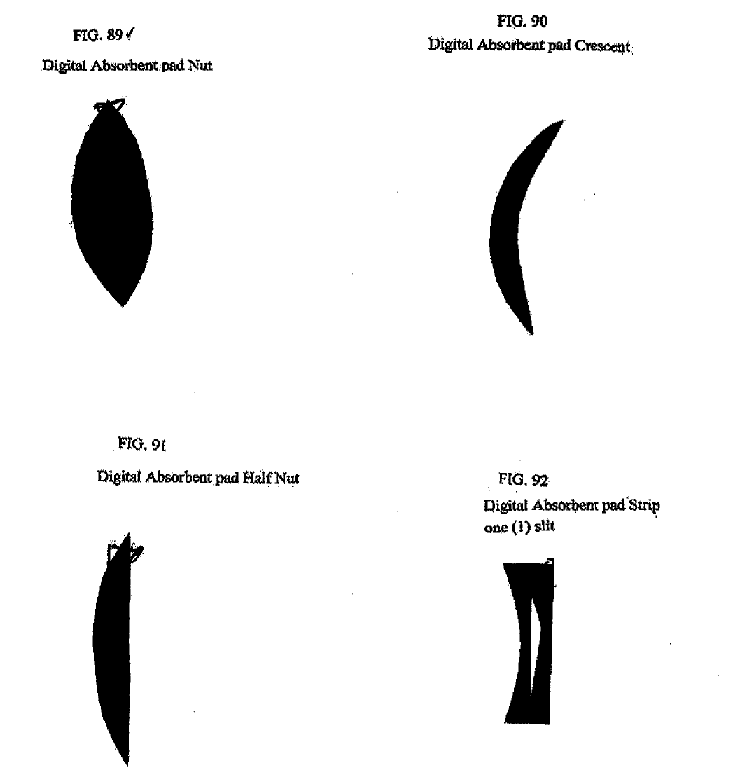

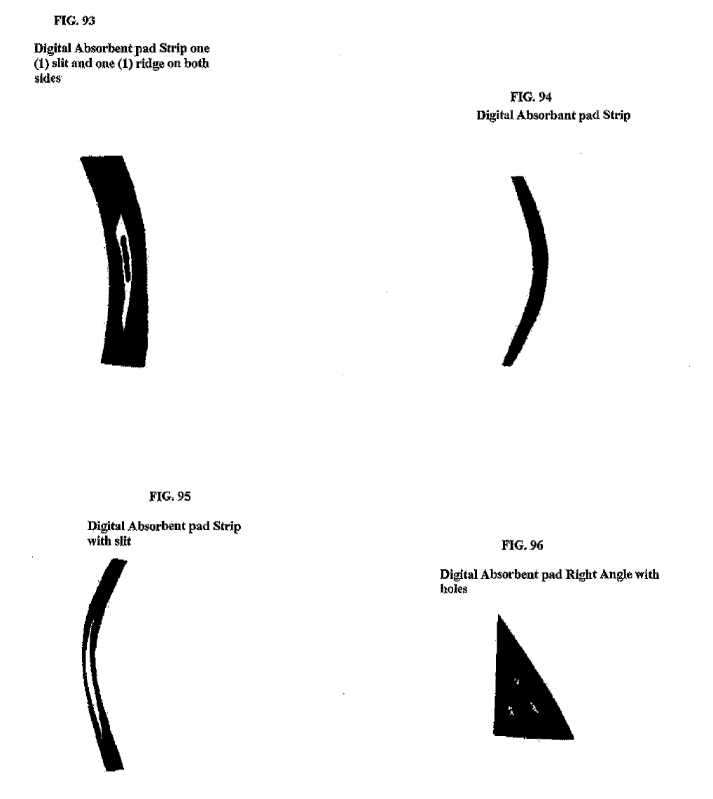

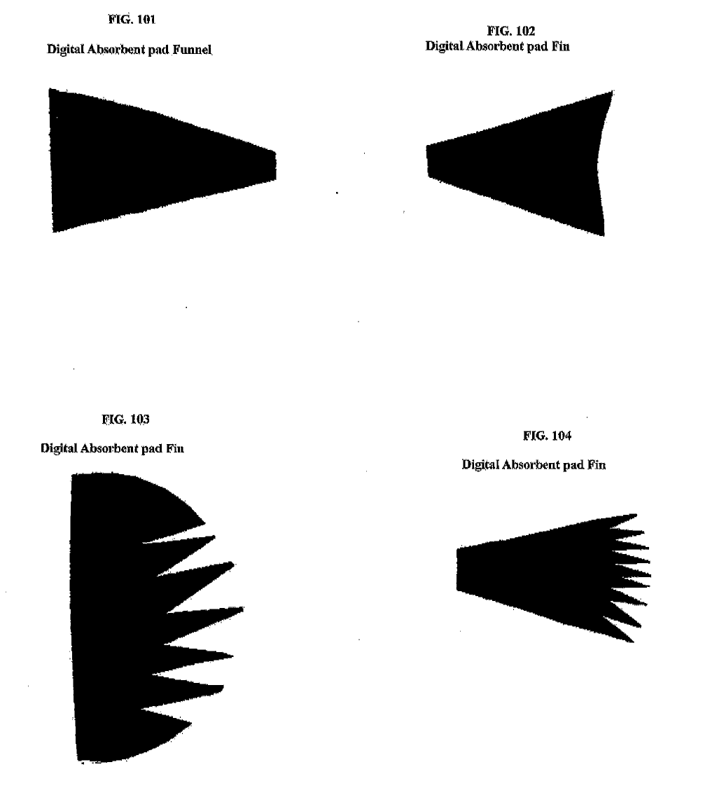

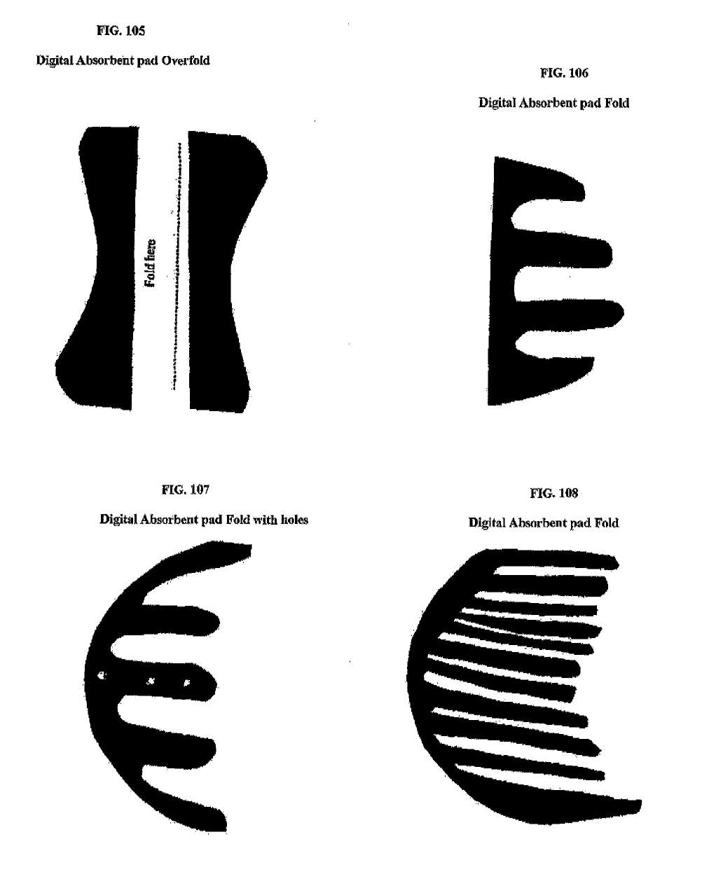

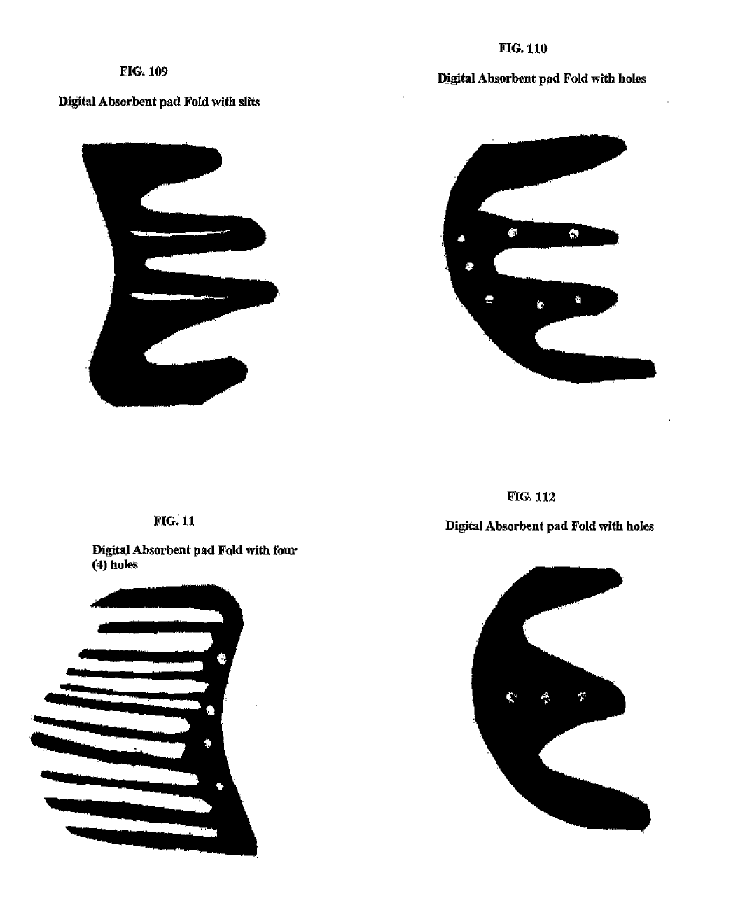

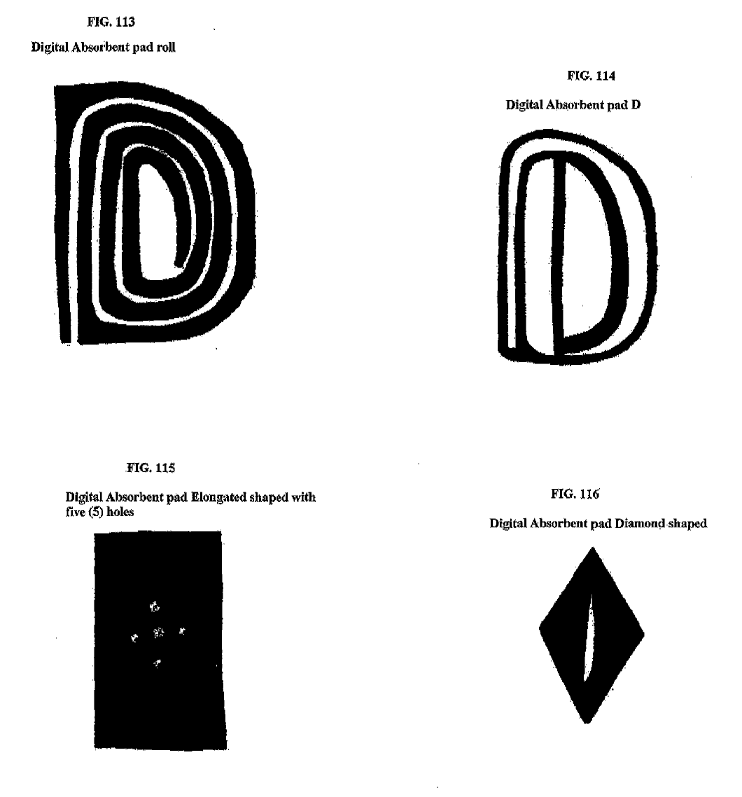

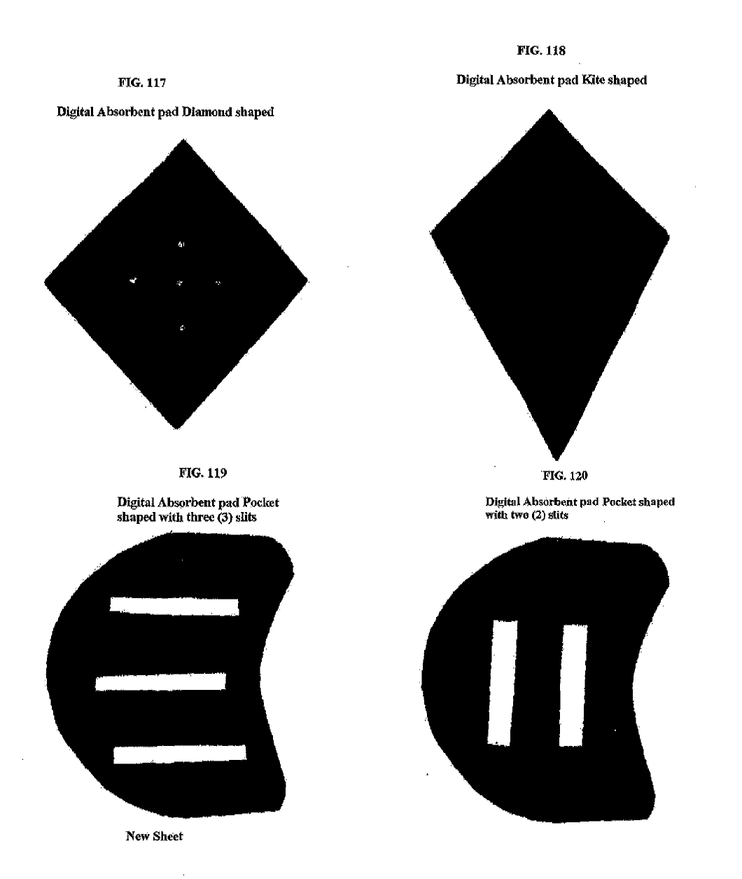

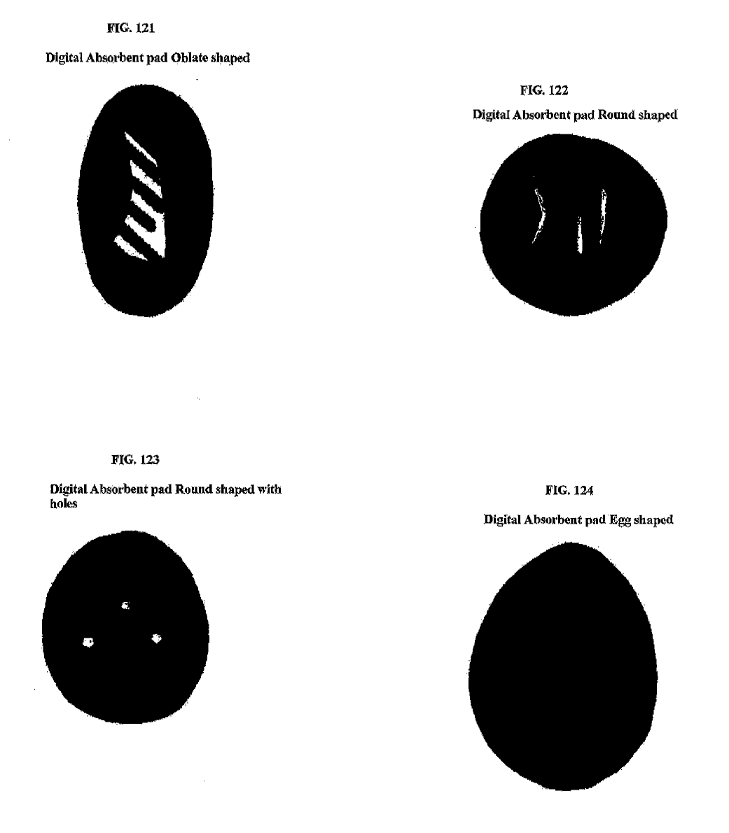

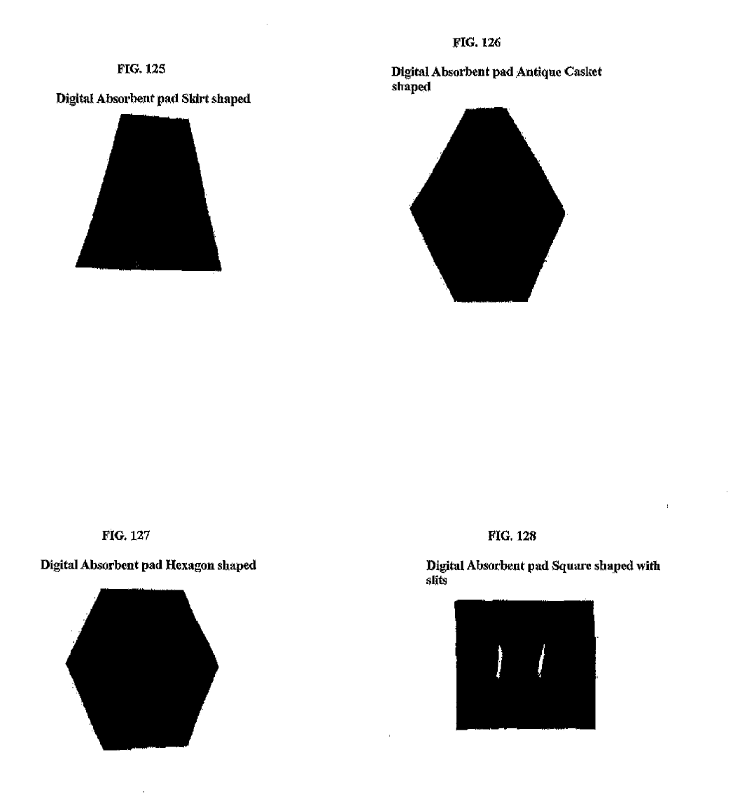

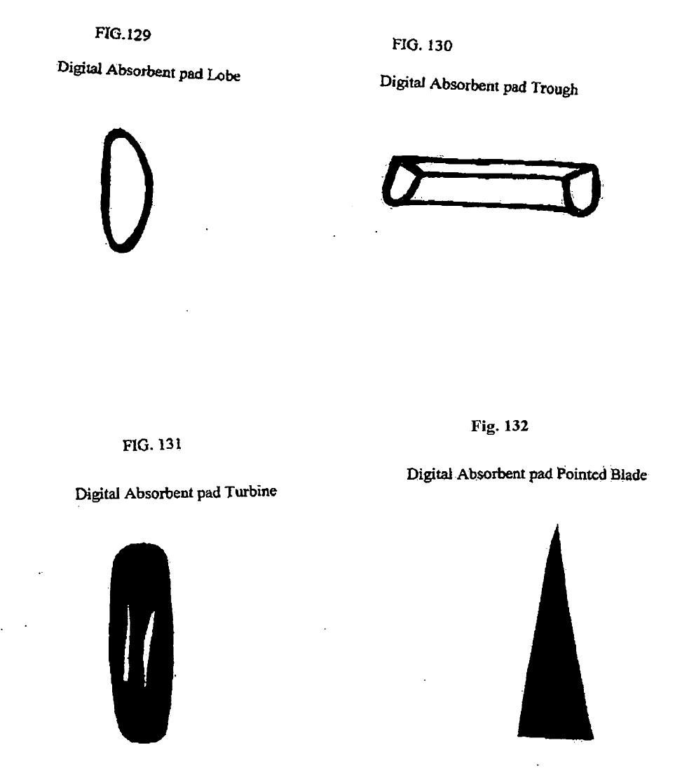

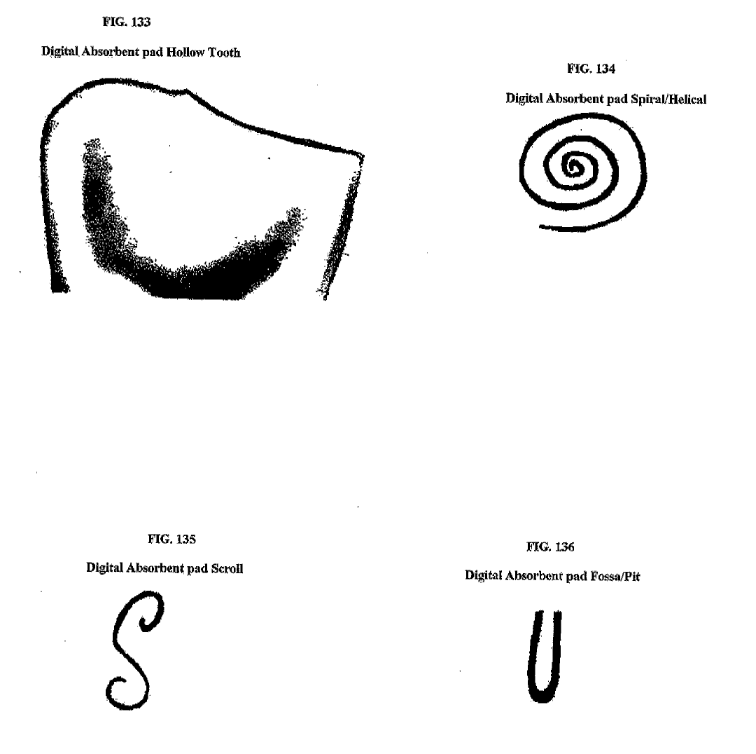

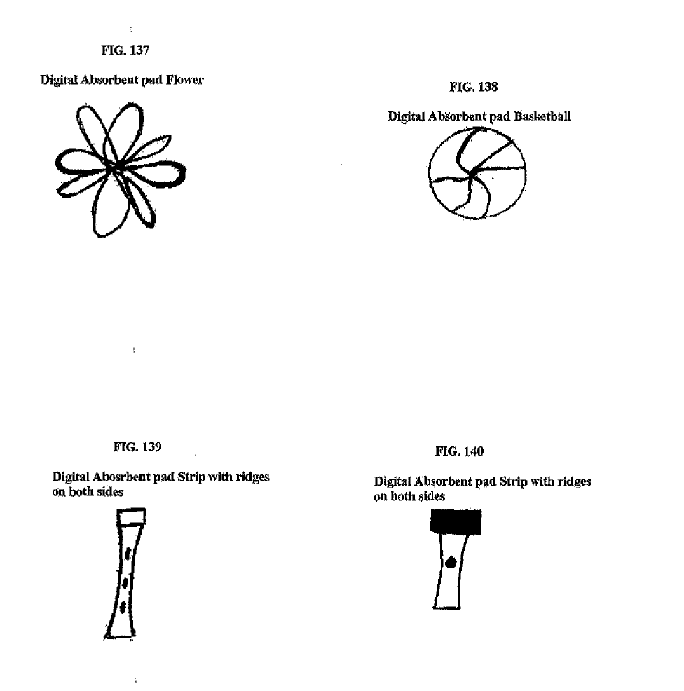

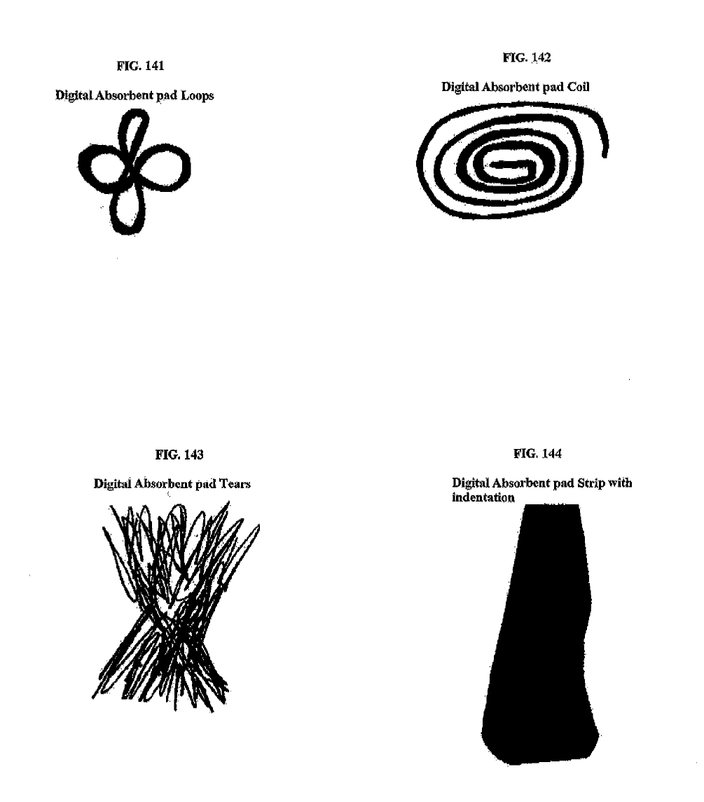

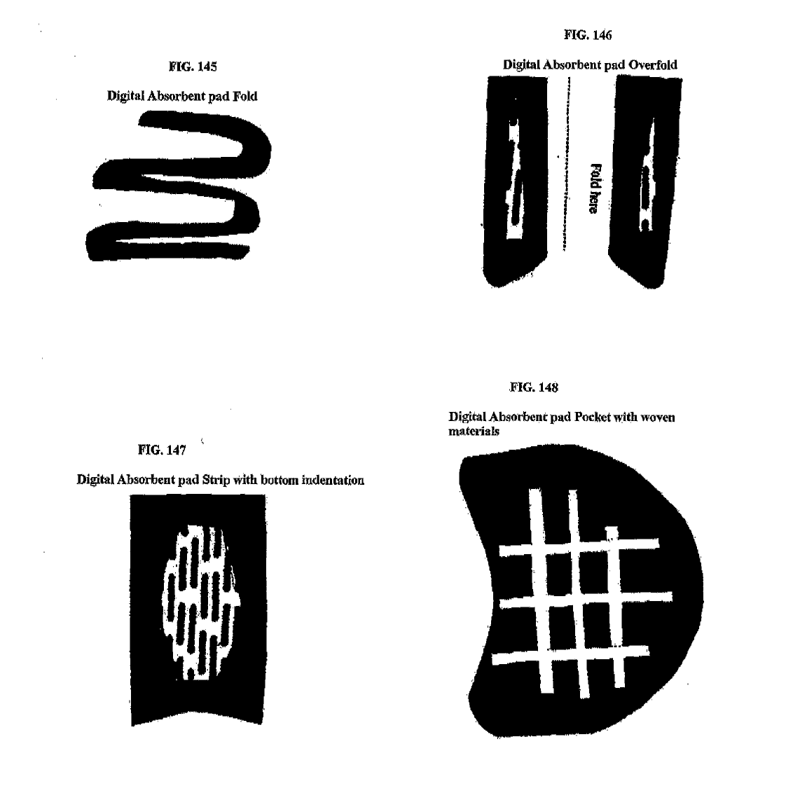

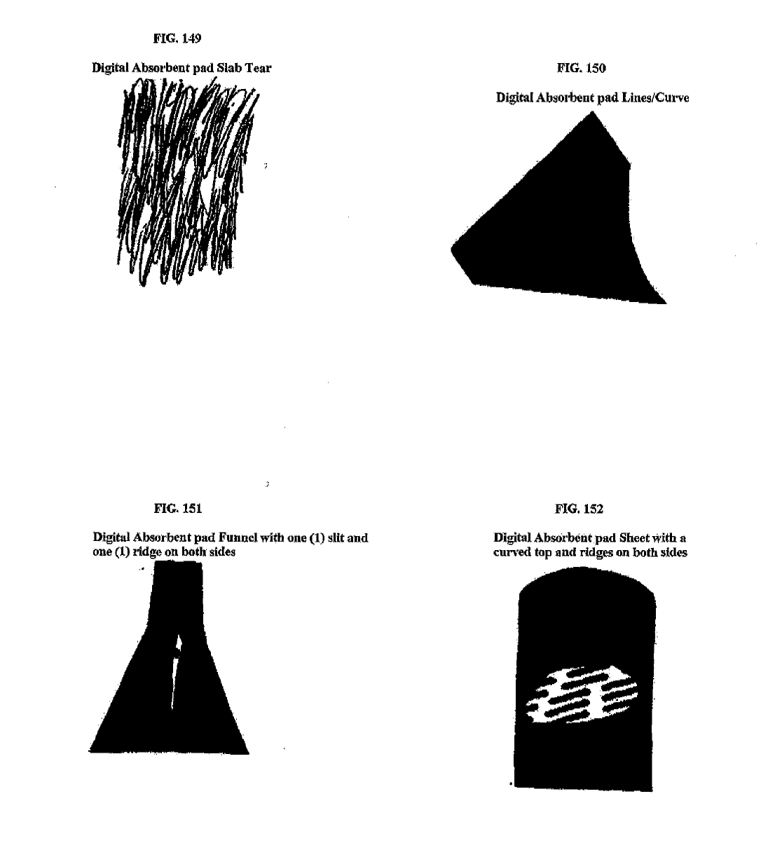

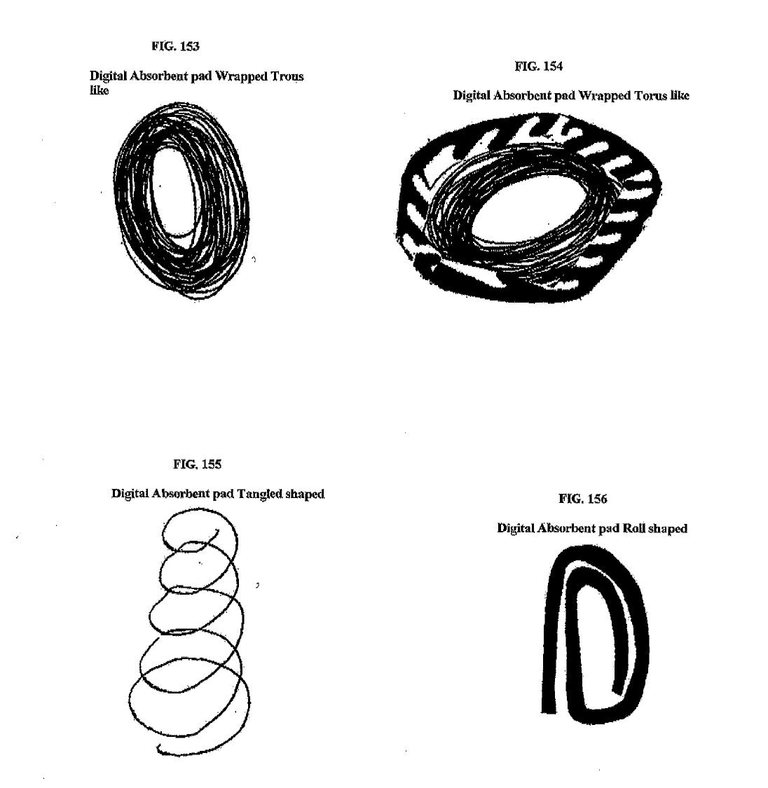

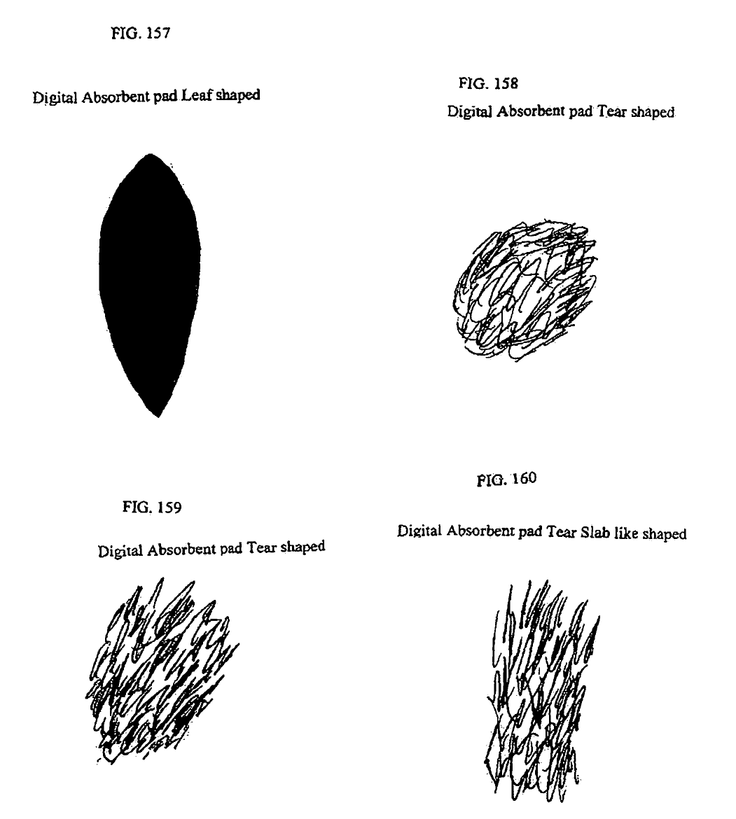

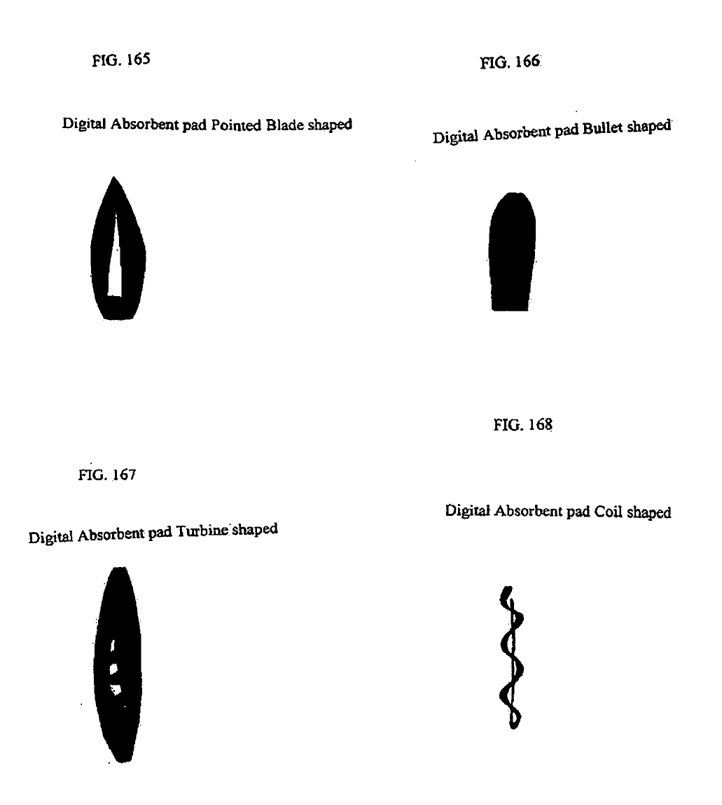

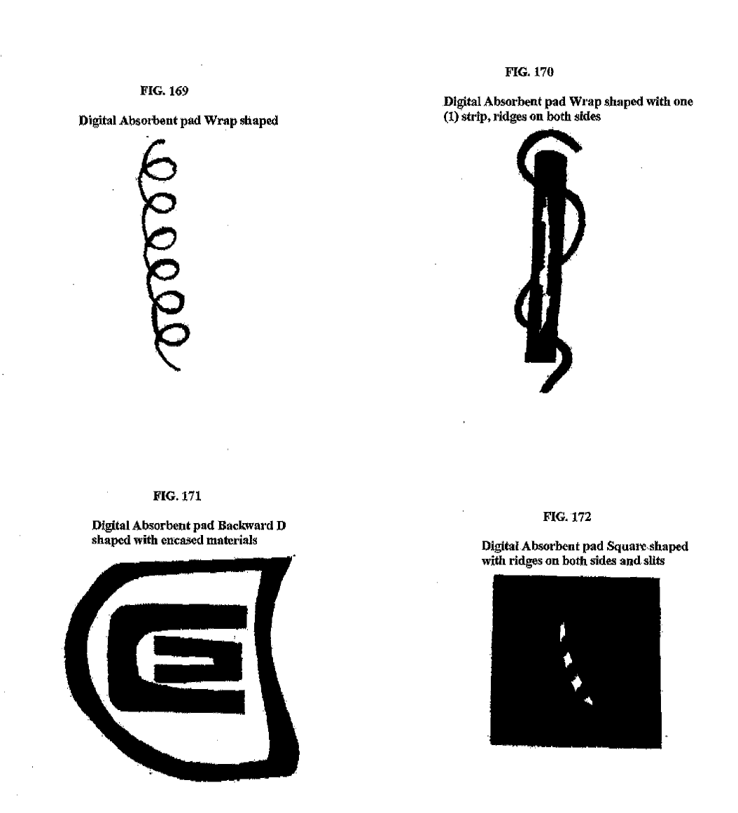

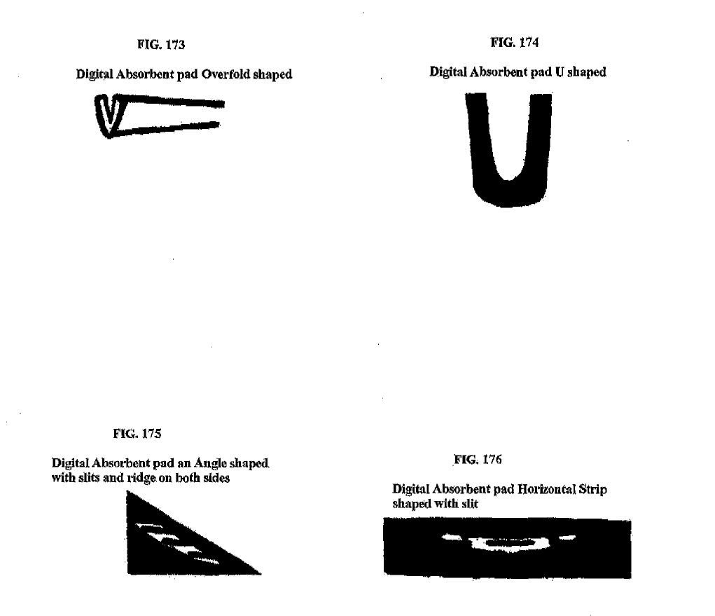

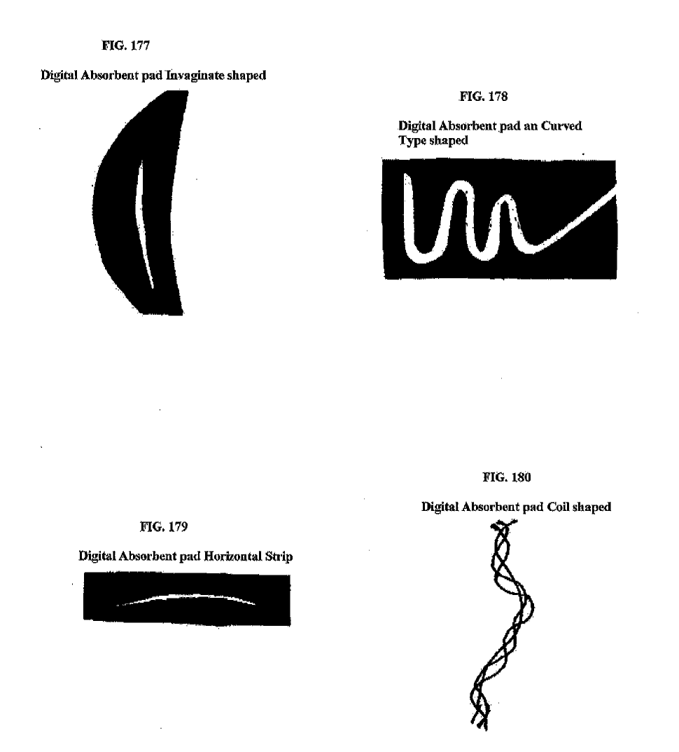

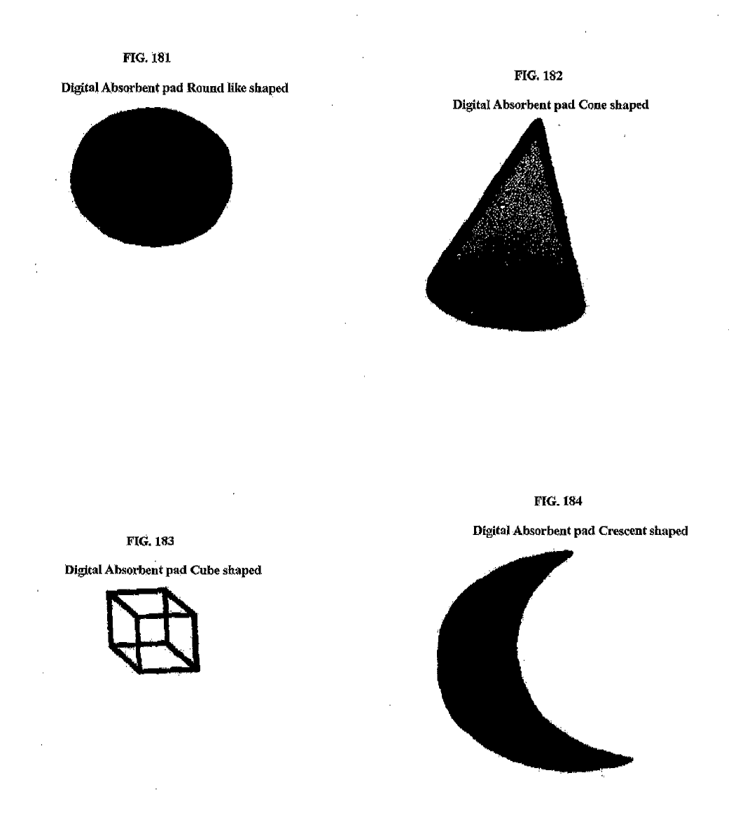

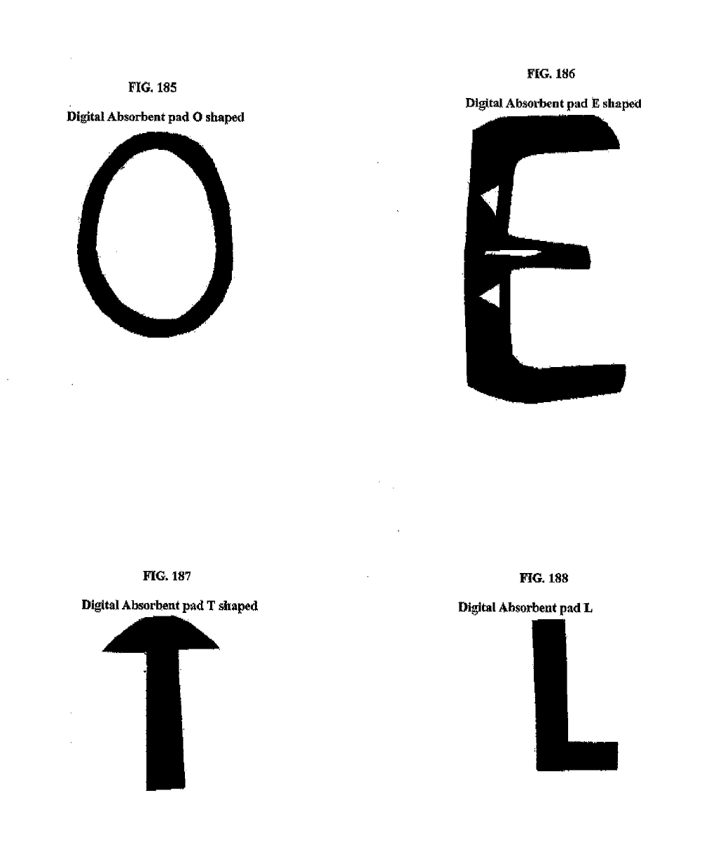

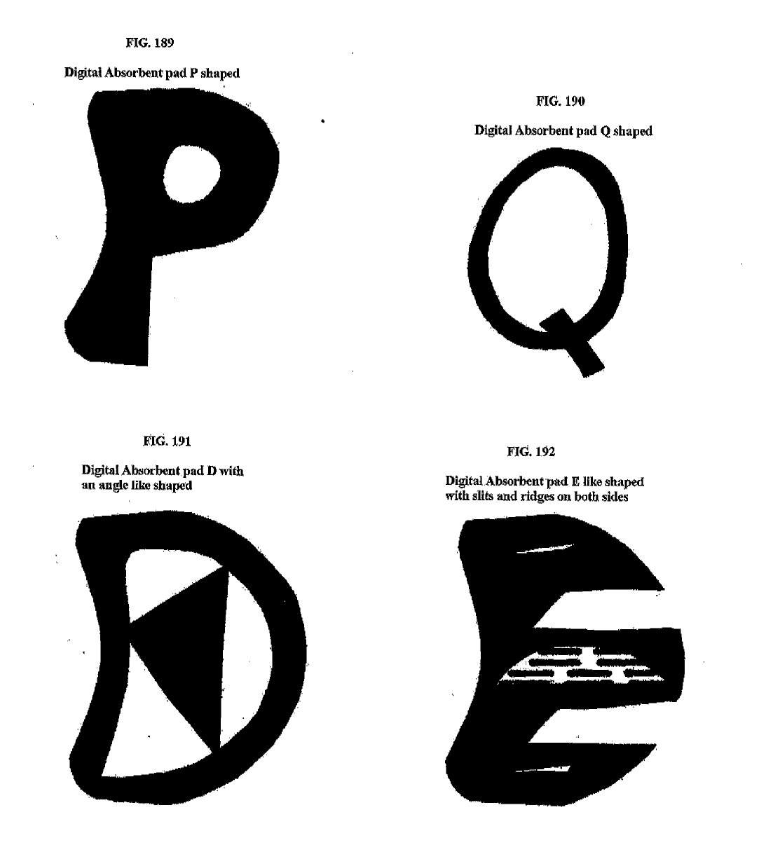

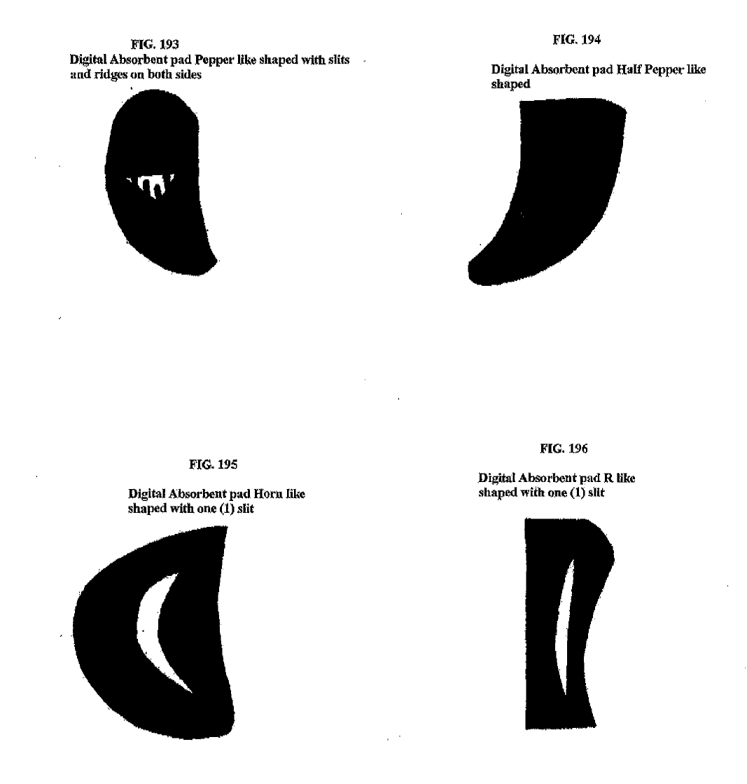

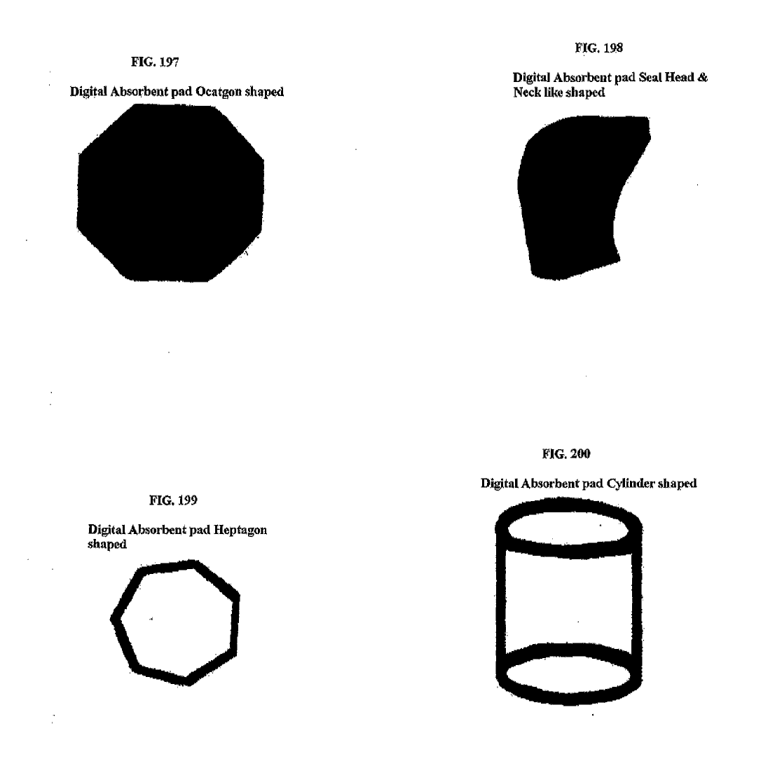

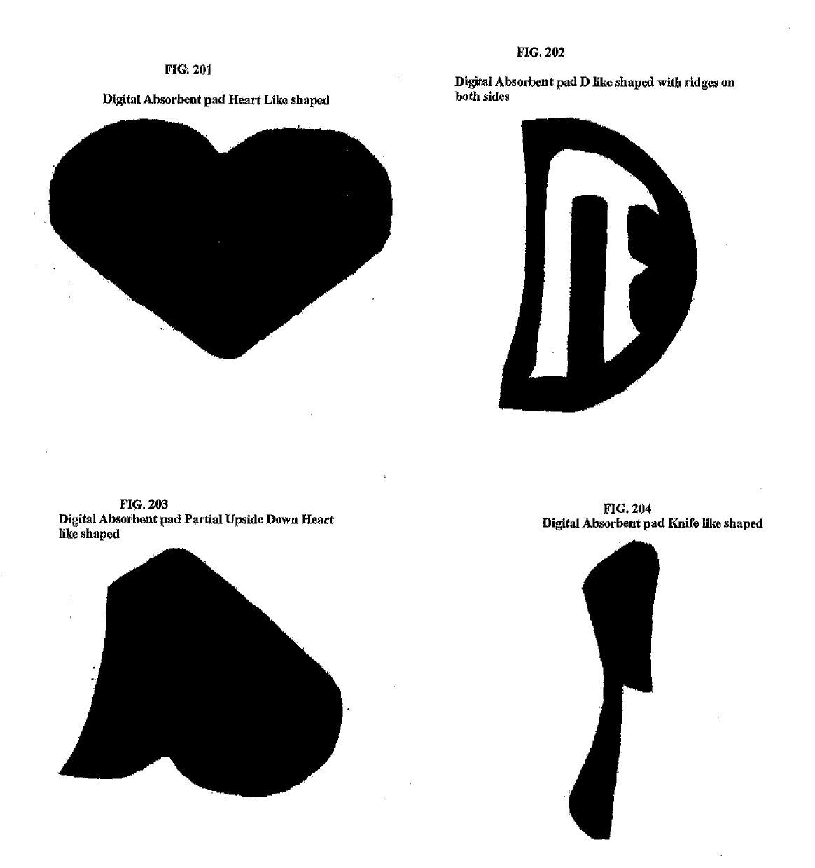

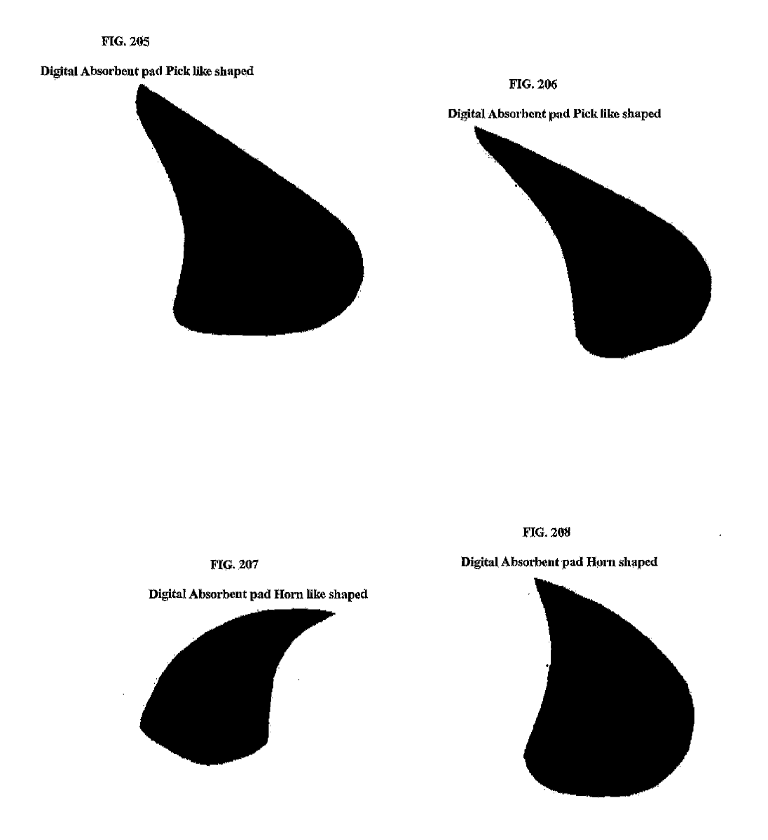

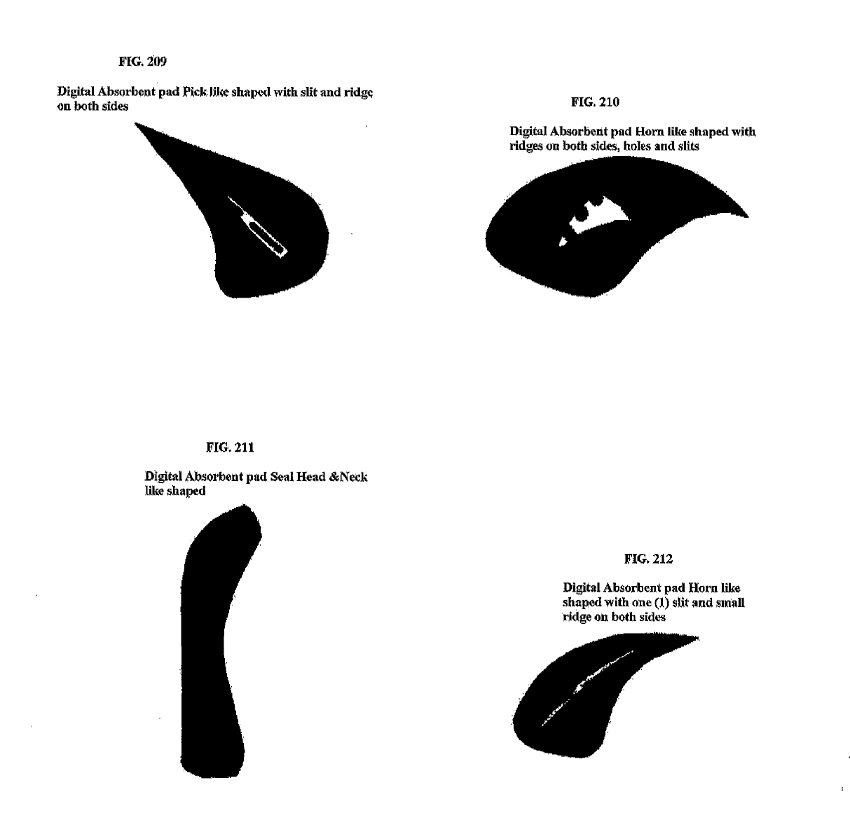

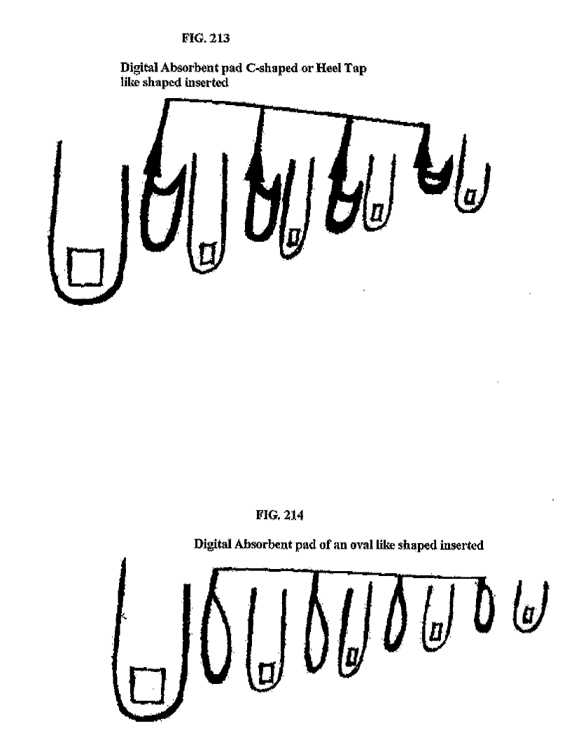

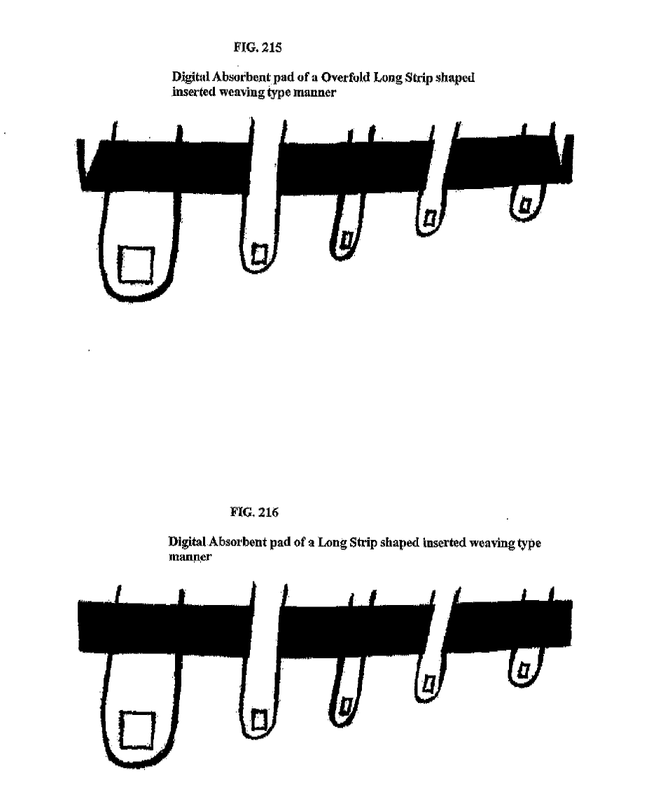

The inter-digital pad can be configured as depicted in FIGS. 85-216. Each pad can be configured as illustrated in FIGS. 85-216 will be configured as shown in FIG. 1A. However the pad can be configured with a single layer of absorbent pad or non-absorbent pad.

What is claimed is:

1. A n inter-digital pad for insertion between the toes of a human foot, for preventing irritation, the pad comprising:
an non-absorbent outer layer encompassing an absorbent inner layer with the outer peripheral edges sealed to form a body;
the body having a rearward portion, a forward portion, a first side and an opposing side;
the body having a polygonal shape consisting of rectangle and square; the rearward portion fitting to a root of the toes of a human foot;
the body being manufactured in a predetermined length, width, and thickness to fit a wearer of the pad.

2. The pad of claim 1 wherein the length ranges up to 10 inches.

3. The pad of claim 1 wherein the width ranges up to 8 inches.

4. The pad of claim 1 wherein the thickness ranges up to 4 inches.

5. The pad of claim 1 wherein the outer section is medicated.

6. The pad of claim 1 wherein the inner section is medicated.

7. The pad of claim 1 wherein the outer layer have a plurality of apertures.

8. The pad of claim I wherein the inner section and the outer section have a plurality of apertures extending therethrough.

9. The pad of claim 1 wherein the outer layer have a plurality of slits.

10. The pad of claim 1 wherein the inner section and the outer section have a plurality of slits extending therethrough.

11. The pad of claim 1 wherein the inner layer is made of an absorbent material consisting of the group of cotton, polyester, sponge material, rayon, cellulose material, absorbent polymers, and any combination thereof

12. The pad of claim I wherein the outer layer is made of a non-absorbent material consisting of the group of non-absorbent polymers, gel material, glass material, synthetic material and any combination thereof.

13. An inter-digital pad for insertion between the toes of a human foot, for preventing irritation, the pad comprising:
an non-absorbent outer layer encompassing an absorbent inner layer with the outer peripheral edges sealed to form a body;
the body having a rearward portion, a forward portion, a first side and an opposing side;
the body having a configuration consisting of: Flash Drive with holes
86: K Like shaped with three (3) holes
87: Spacer with holes
88: Comb
89: Nut
90: Crescent
91: HalfNut
92: Strip one (1) slit
93: Strip one (1) slit and one (1) ridge on both sides
94: Strip
95: Strip with slit
96: Right Angle with holes
101: Funnel
102: Fin
103: Fin
104: Fin
105: Overfold
106: Fold
107: Fold with holes
108: Fold
109: Fold with slits
110: Fold with holes
111: Fold with four (4) holes
112: Fold with holes
113: Roll
114: D
115: Elongated shaped with five (5) holes
116: Diamond shaped
117: Diamond shaped
118: Kite shaped
119: Pocket shaped with three (3) slits
120: Pocket shaped with two (2) slits
121: Oblate shaped
122: Round shaped
123: Round shaped with holes
124: Egg shaped
125: Skirt shaped
126: Antique Casket shaped
127: Hexagon shaped
128: Square shaped with slits
129: Lobe
130: Trough
131: Turbine
132: Pointed Blade
133: Hollow Tooth
134: SpirallHelical
135: Scroll
136: FossalPit
137: Flower
138: Basketball
139: Strip with ridges on both sides
140: Strip with ridges on both sides
141: Loops
142: Coil
143: Tears
144: Strip with indentation
145: Fold
146: Overfold
147: Strip with bottom indentation and with ridges on both sides
148: Pocket with woven materials
149: Slab Tear
150: Lines/Curve
151: Funnel with one (1) and one (1) ridge on both sides
152: Sheet with a curved top and ridges on both sides
153: Wrapped Torus like
154: Wrapped Torus like
155: Tangled shaped
156: Roll shaped
157: Leaf shaped
158: Tear shaped
159: Tear shaped
160: Tear Slab like shaped
165: Pointed Blade shaped
166: Bullet shaped
167: Turbine shaped
168: coil shaped
169: Wrap shaped
170: Wrap shaped with one (1) strip, rides on both sides
171: Backward D shaped with encased materials
172: Square shaped with ridges on both sides and slits
173: Overfold shaped
174: U shaped
175: Angle shaped with slits and ridge on both sides
176: Horizontal Strip shaped with slit
177: Invaginate shaped
178: Curved Type shaped
179: Horizontal Strip
180: Coil shaped
181: Round like shaped
182: Cone shaped
183: Cube shaped
184: Crescent shaped
185: 0 shaped
186: E shaped
187: T shaped
188: L shaped
189: P shaped
190: Q shaped
191: D with an angle like shaped
192: E like shaped with slits and ridges on both sides
193: Pepper like shaped with slits and ridges on both sides
194: Half Pepper like shaped
195: Horn like shaped with one (1) slit
196: R like shaped with one (1) slit
197: Octagon shaped
198: Seal Head & Neck like shaped
199: Heptagon shaped
200: Cylinder shaped
201: Heart Like shaped
202: D like shaped with ridges on both sides
203: Partial Upside Down Heart like shaped
204: Knife like shaped
205: Pick like shaped
206: Pick like shaped
207: Horn like shaped
208: Horn like shaped
209: Pick like shaped with slit and ridge on both sides
210: Horn like: shaped with ridges on both sides, holes and slits
211: Seal Head & Neck like shaped
212: Horn like shaped with one (1) slit and small ridge on both sides
213: C-shaped or Heel Tap like shaped inserted
214: oval like shaped. inserted
215: Overfold Long Strip shaped inserted weaving type manner
216: Long Strip shaped inserted weaving type manner;
the rearward portion fitting to a root of the toes of a human foot;
the body being manufactured in a predetermined length, width, and thickness to fit a wearer of the pad.
